(19) United States
(12) Patent Application Publication
(10) Pub. No.: US 2024/0058043 A1
(43) Pub. Date: Feb. 22, 2024

(54) BONE FIXATION IMPLANTS

(71) Applicant: WRIGHT MEDICAL TECHNOLOGY, INC., Memphis, TN (US)

(72) Inventor: John Kent ELLINGTON, Charlotte, NC (US)

(73) Assignee: WRIGHT MEDICAL TECHNOLOGY, INC., Memphis, TN (US)

(21) Appl. No.: 18/501,390

(22) Filed: Nov. 3, 2023

Related U.S. Application Data
(63) Continuation of application No. 17/293,807, filed on May 13, 2021.
(60) Provisional application No. 62/785,460, filed on Dec. 27, 2018.

(51) Int. Cl. A61B 17/80

(52) U.S. Cl. CPC A61B 17/8095; A61B 17/8004; A61B 2017/00867

(57) ABSTRACT

Improved shape memory material orthopedic fixation implant embodiments useful for various joint fusion procedures are disclosed.

### CROSS-REFERENCE TO RELATED APPLICATIONS

This is a continuation of U.S. patent application Ser. No. 17/293,807, filed May 13, 2021, which is a National Stage Application, filed under 35 U.S.C. 371, of International Patent Application No. PCT/US2019/068567, filed on Dec. 26, 2019, which claims priority to U.S. Provisional Patent Application No. 62/785,460, filed on Dec. 27, 2018, the entireties of which are incorporated herein by reference.

### FIELD OF DISCLOSURE

The present disclosure relates to the field of orthopedic staples.

### BACKGROUND

Because of the shape memory properties, Nitinol has various uses throughout medicine, including cardiac stents, orthodontic wires, and musculoskeletal implants. Early generation Nitinol staples released in the 1990s and early 2000s were limited by the need for refrigeration to maintain the non-compressed state and/or heating (i.e. electrocautery) to reach the compressed state. New generation Nitinol staples have gained popularity as a musculoskeletal implant as a result of ease and speed of insertion as well as super-elastic properties at human body temperature, allowing implants to deform and return to their original shape, i.e. with weightbearing. Nitinol staples also undergo a thermal conformational from a non-compressed state to a compressed state with body heat, making them ideal for use in midfoot and hindfoot fusions, as well as certain trauma applications. Nitinol staples maintain continuous compression, and, as a result, will actively reduce and compress bone fragments together as bone resorption occurs. In vitro biomechanical testing has shown that Nitinol staples maintain time zero contact force and contact area after mechanical loading, and had a 7% increase in compression in the first 10 minutes after implantation, unlike plate and screw constructs. The staple design also offers four points of fixation in bone fragments, unlike two points with screws. Nitinol staples also do not require bicortical fixation because the compression footprint extends beyond the tips of the staple legs. Clinically, the new generation of Nitinol staples were found to have a high union rate of 92.7% for both midfoot and hindfoot arthrodesis with no significant difference between staple and screw vs. staple alone constructs.

Current Nitinol staples consist of two or multi-leg devices with in-line compression and are meant to be used without discrimination across joint/bone surfaces.

Thus, there is a continuing need for an improved external fixator that can provide such adaptability.

### SUMMARY

According to an embodiment, an orthopedic implant is disclosed that comprises: a bridge including a first end and a second end; a first leg extending from the first end of the bridge; a second leg extending from the second end of the bridge; the bridge comprising a metal spacer structure provided in between the first leg and the second leg and extending from the bridge in the same general direction as the first and second legs, wherein the metal spacer structure is configured to be inserted between two osteotomy bone surfaces.

An orthopedic implant according to another embodiment is disclosed. The orthopedic implant comprises: a bridge including a first end and a second end, wherein each of the first and second ends of the bridge is provided with one or more holes for receiving bone screws; and a first leg and a second leg provided between the first end and the second end and extending from the bridge.

An orthopedic implant according to another embodiment is disclosed. The orthopedic implant comprises: a bridge including a first end and a second end; a first leg extending from the first end of the bridge; a second leg extending from the second end of the bridge; where the at least the bridge portion of the orthopedic implant is made of a shape memory material such that the bridge is movable between an insertion shape and an implanted shape, where when in the insertion shape the bridge is straight, and when in the implanted shape the bridge is bowed in a direction opposite from the extension direction of the first and second legs and urges the first and second legs toward each other.

An orthopedic implant according to another embodiment is disclosed. The orthopedic implant comprises: a bridge having a longitudinal axis and including a first end and a second end; a bridge extension provided at each of the first end and the second end, wherein the bridge extensions extend in a transverse direction with respect the longitudinal axis of the bridge; and a plurality of legs extending from each of the bridge extensions; where the orthopedic implant being made of a shape memory material such that the orthopedic implant is movable between an insertion shape and an implanted shape, where in the implanted shape, the plurality of legs are urged in more than one preset directions to produce compression load in the more than one preset directions.

According to some embodiments, a shape memory material orthopedic staple implant that provides improved rotational stability and improved compression in cancellous bone is disclosed. The improved orthopedic staple implant is particularly useful for subtalar fusion. Unlike other orthopedic staples, all portions of the orthopedic staple implant of this embodiment comprises a ribbon or blade-like dimension and all portions have a width that is substantially greater than their thickness.

### BRIEF DESCRIPTION OF THE DRAWINGS

The inventive concepts of the present disclosure will be described in more detail in conjunction with the following drawing figures. The structures in the drawing figures are illustrated schematically and are not intended to show actual dimensions.

FIGS. 1A-1E are illustrations of metal-spacer hybrid staples examples according to some embodiments.

FIGS. 2A-2B are illustrations of staple-plate hybrid orthopedic implants according to some embodiments.

FIGS. 3A-3B are illustrations of an orthopedic staple according to another embodiment.

FIGS. 4A-4B are illustrations of an orthopedic staple that provides multi-planar compression according to some embodiments.

FIGS. 5A-5D are illustrations of an orthopedic staple that provides improved rotational stability and improved compression in cancellous bones according to some embodiments.

FIGS. 6A-6E are illustrations of varioius additional embodiments of orthopedic staple implants that provide improved rotational stability and improved compression in cancellous bones.

FIGS. 7A-7E are illustrations showing various features of an orthopedic staple implant whose bridge portion between the staple legs is configured to be more like a bone plate.

FIGS. 8A-10I are illustrations of various examples of blade/plate hybrid orthopedic fixation implants according to the present disclosure.

FIG. 11 is a illustration of an embodiment of a shape memory material orthopedic fixation implant that is configured for use in TMT joint fusion procedure.

FIGS. 12A-12B are illustrations of an embodiment of a shape memory material orthopedic fixation implant that is particularly configured for use in navicular-cuneiform fusion procedure.

FIG. 13 is a illustration of an embodiment of a shape memory material orthopedic fixation implant that is particularly configured for use in subtalar fusion procedure.

FIGS. 14A-14C are illustrations of a shape memory orthopedic staple implant embodiment that is configured for lesser metatarsal osteotomy fusion.

FIGS. 15A-15C are illustrations of a shape memory material orthopedic staple implant embodiment that is particularly configured for use in hallux metatarsophalangeal joint fusion procedure.

FIGS. 16A-17B are illustrations of two embodiments of a shape memory material orthopedic staple implant that is particularly configured for fixating a Jones fracture.

FIG. 18 is a illustration showing another embodiment of a staple implant that is configured to match the anatomic contour around a TMT joint.

FIGS. 19A-19B are illustrations of another embodiment of a staple implant that is configured for hallux metatarsophalangeal fusion.

FIGS. 20A-20B are illustrations of a staple implant according to another embodiment. The implant is configured to be particularly useful in subtalar fusion procedure.

FIGS. 21A-21D are illustrations of additional embodiments of a hybrid implant comprising a bone plate portion.

FIGS. 22A-22F are illustrations describing a fusion guide instrument that can be used for aiding subtalar fusion procedure according to another aspect of the present disclosure.

FIGS. 23A-23B are illustrations describing a compression device for orthopedic staples.

### DETAILED DESCRIPTION

This description of the exemplary embodiments is intended to be read in connection with the accompanying drawings, which are to be considered part of the entire written description. The drawing figures are not necessarily to scale and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. When only a single machine is illustrated, the term “machine” shall also be taken to include any collection of machines that individually or jointly execute a set (or multiple sets) of instructions to perform any one or more of the methodologies discussed herein. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

Unless otherwise expressly stated, it is in no way intended that any method set forth herein be construed as requiring that its steps be performed in a specific order, nor that with any apparatus, specific orientations be required, unless specified as such. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

In this disclosure, the shape memory material for the disclosed orthopedic implants such as staples, hybrid staples, blades, etc., can be made of any of the known shape memory materials such as shape memory alloys such as Nitinol that are suitable for orthopedic applications that maintains the pre-deformed (i.e. remembered) shape at the body temperature of the orthopedic patient in which the disclosed orthopedic implants are implanted.

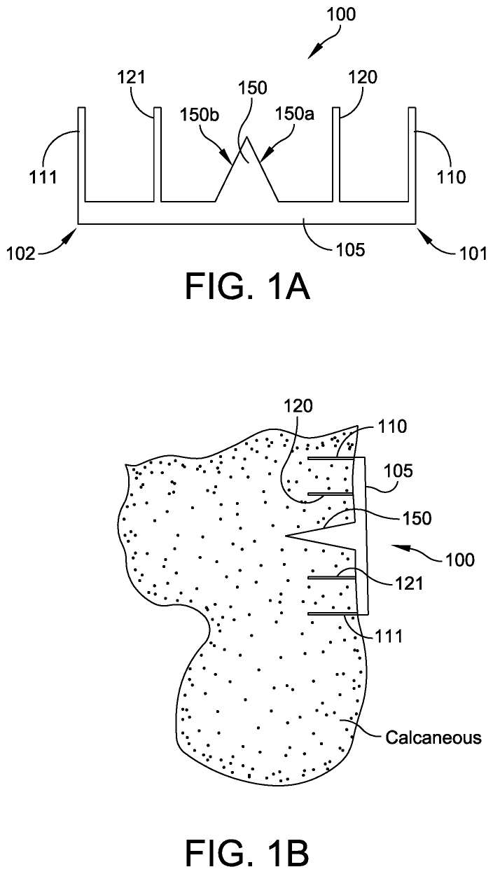

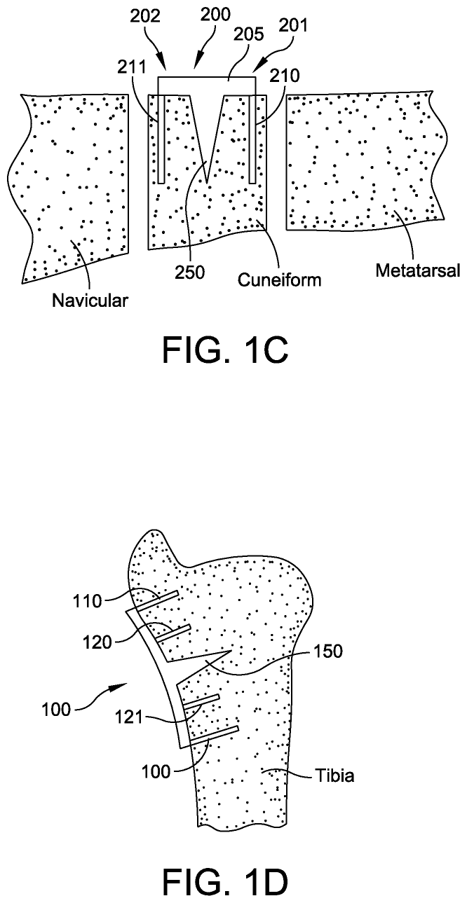

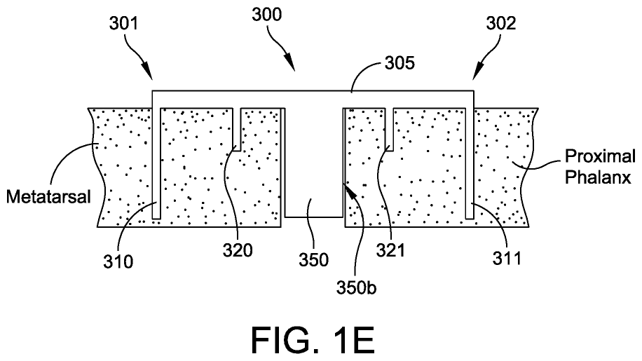

Referring to FIGS. 1A-1E, orthopedic implants according to some embodiments of the present disclosure are disclosed. The orthopedic implants have a metal-spacer staple hybrid structure that incorporates metal spacer feature into a shape memory metal orthopedic staples. These hybrid staples are useful in such procedures as Evans osteotomy, Cotton osteotomy, and tibial osteotomy where the metal spacer structure of the staple takes the place of the spacers fashioned from a piece of bone and being combined into a staple structure provides the combined ability to keep the spacer secured between the two osteotomy bone surfaces. These hybrid staples can also be useful in other osteotomies such as in femur, hip, etc.

FIG. 1A is a schematic side view of a metal-spacer hybrid orthopedic staple 100 comprising a bridge 105 including first and second ends 101, 102, respectively. The staple 100 comprises a first set of legs 110, 111. The first leg 110 extends from the first end 101 of the bridge. The second leg 111 extends from the second end 102 of the bridge. The metal-spacer hybrid staple 100 comprises a metal spacer structure 150 provided along the bridge 105 between the first and second legs 110, 111. The metal spacer structure 150 extends in the same general direction as the legs 110 and 111. The particular location of the metal spacer structure 150 on the bridge 105 can be selected to be an appropriate location for the particular osteotomy application. For example, the metal spacer structure 150 can be located in the center of the bridge 105 equidistant from the first and second legs 110, 111 or the metal spacer structure 150 can be located on the bridge off-center so that it is closer to one of the two legs 110, 111 than the other.

In some embodiments, the metal spacer structure 150 can have a triangular wedge shaped profile having two planar surfaces 150a, 150b that face the first and second legs. The embodiments of the orthopedic implant examples 100 and 200 shown in FIGS. 1A-1D have such triangular wedge shaped meal spacer structures 150 and 250. In some other embodiments, the metal spacer structure can have a trapezoidal shaped profile and have two planar surfaces facing the first and second legs. The embodiment of the orthopedic implant example 300 shown in FIG. 1E has such trapezoidal shaped metal spacer structure 350.

In some embodiments, the metal-spacer hybrid orthopedic staple 100 can further comprise additional sets of legs as necessary. In the illustrated example in FIG. 1A, the orthopedic staple 100 further comprises a second set of legs 111, 121. The first leg 120 of the second set of legs is located between the first leg 110 of the first set and the metal wedge structure 150. The second leg 121 of the second set of legs is located between the second leg 111 and the metal spacer structure 150. The additional sets of legs can provide additional compression force or distraction force depending on the manner in which the orthopedic staple 100 is preset.

FIG. 1B is a schematic illustration showing the metal-spacer hybrid orthopedic staple 100 in an Evans osteotomy application. The metal-spacer hybrid orthopedic staple 100 is implanted into a calcaneus bone structure over an Evans osteotomy. The legs 110, 111, 120, 121 of the metal-spacer hybrid staple 100 are implanted on both sides of the Evans osteotomy and the metal spacer structure 150 is wedged into the Evans osteotomy. The metal-spacer hybrid orthopedic staple 100 is made of a shape memory alloy and in this illustrated example, the staple 100 is preconditioned to apply compression load to the Evans osteotomy and the metal spacer structure 150.

FIG. 1C is a schematic illustration showing a metal-spacer hybrid orthopedic staple 200 according to another embodiment. The orthopedic staple 200 as shown is being used in a Cotton osteotomy application. The metal-spacer hybrid orthopedic staple 200 is implanted into a cuneiform bone over a Cotton osteotomy. The orthopedic staple 200 comprises a bridge 205 including first and second ends 201, 202, respectively. The staple 200 comprises a first set of legs 210, 211. The first leg 210 extends from the first end 201 of the bridge. The second leg 211 extends from the second end 202 of the bridge 205. The metal-spacer hybrid staple 200 comprises a metal spacer structure 250 provided along the bridge 205 between the first and second legs 210, 211 and extends in the same general direction as the legs 210 and 211. The location of the metal spacer structure 250 on the bridge 205 is selected to be an appropriate location for the particular osteotomy application.

FIG. 1D is a schematic illustration showing the metal-hybrid hybrid orthopedic staple 100 implanted in a tibial osteotomy application according to another embodiment. The legs 110, 111, 120, 121 are implanted into a tibia with the metal spacer structure 150 wedged into an osteotomy cut into the tibia.

FIG. 1E is a schematic illustration of a metal-spacer hybrid orthopedic staple 300 according to another embodiment. The orthopedic staple 300 comprises a bridge 305, a first end 301, and a second end 302. A first leg 310 extends from the first end 301. A second leg 311 extends from the second end 302. The orthopedic implant 300 also comprises a metal spacer structure 350 provided along the bridge 305 between the first and second legs 310, 311 and extends in the same general direction as the legs 310, 311. The location of the metal spacer structure 350 on the bridge 305 is selected to be an appropriate location for the specific application. The orthopedic staple 300 can further comprise a second set of legs 320, 321.

In the illustrated example in FIG. 1E, the staple 300 further comprises a second set of legs 320, 321. The first leg 320 of the second set of legs is located between the first leg 310 of the first set and the metal spacer structure 350. The second leg 321 of the second set of legs is located between the second leg 311 and the metal spacer structure 350.

In the illustrated example in FIG. 1E, the staple 300 is implanted into a metatarsal-phalanx joint. The legs 310, 311, 320, 321 of the metal-spacer hybrid staple 300 are implanted on both sides of the metatarsal-phalanx joint and the metal spacer structure 350 is wedged between the metatarsal and the phalanx bone. The metal-spacer hybrid orthopedic staple 300 is made of a shape memory alloy and in this illustrated example, the staple 300 is preconditioned to apply a distraction force keeping the metatarsal-phalanx joint space open to receive the metal spacer structure 350. As shown in FIG. 1E, the metal spacer structure 350 can have a trapezoid shaped profile that is wider near the bridge 305 and tapers thinner away from the bridge 305. In some embodiments, the metal spacer structure 350 can have two parallel sides having a square or a rectangular profile. Preferably, the metal spacer structure 350 comprises two planar surfaces 350a, 350b that face the first and second legs 310, 311.

In some embodiments, the portions of the metal-spacer hybrid staples 100, 200, and 300 forming the metal spacer structures 150, 250, 350, respectively, can have porous or perforated structures to promote bone growth into the metal spacer structures.

The metal-spacer hybrid staples can also be usefully applied in distraction arthrodesis procedures such as hallux arthrodesis MTP fusion, calcaneous cuboid fusion, etc.

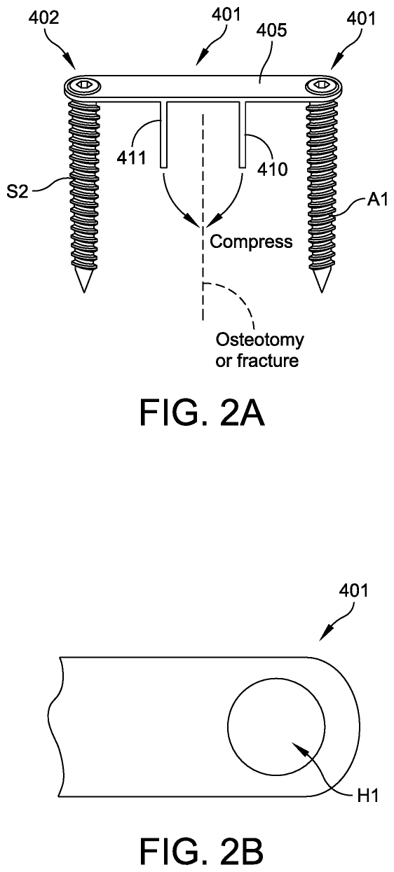

FIG. 2A is an illustration of a staple-plate hybrid orthopedic implant 400 according to another aspect of the present disclosure. The staple-plate hybrid orthopedic implant 400 comprises a bridge 405 having a first end 401 and a second end 402. The bridge 405 of this hybrid orthopedic implant 400 is structured like a bone plate and the two ends 401, 402 are provided with one or more holes for receiving bone screws S1, S2, etc.

In the illustrated example, the two ends 401, 402 of the hybrid orthopedic implant 400 each comprises one hole for receiving a bone screw. FIG. 2B shows a top-down view of the first end 401 of the hybrid orthopedic implant 400 as an example. The first end 401 is provided with a hole H1 for receiving the bone screw S1. In some embodiments, the holes for the bone screws can be threaded to accommodate the threaded heads of locking screws. The threaded screw holes can be configured to accommodate a uni-axial locking screws or poly-axial locking screws. The bone screws S1, S2 can be locking screws, non-locking screws, or compression screws.

The hybrid orthopedic implant 400 further comprises one or more pairs of staple legs 410, 411 positioned between the two ends 401, 402. In the example shown in FIG. 2A, one pair of staple legs 410 and 411 are shown. The hybrid orthopedic implant 400 is made of a shape memory material and in this illustrated example, the hybrid orthopedic staple 400 is preconditioned to apply compression load to the osteotomy or fracture 490. The hybrid orthopedic implant 400 provides fusion site compression with staple legs 410, 411 on the interior while providing bicortical locked or non-locked fixation on perimeter of the construct. The insertion of the staple legs into the compression site would follow the existing shape memory material staple procedure then after the insertion, the bone plate-like bridge portion of the hybrid staple is fixated using bone screws S1, S2.

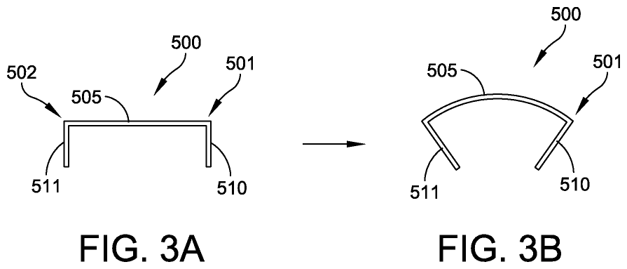

Referring to FIGS. 3A and 3B, a beneficial feature that can be incorporated into any shape memory material orthopedic staples is to have the bridge of the staple preconditioned to bow as shown in FIG. 3B to aid in the compression function of the staple. FIG. 3A shows a shape memory material staple 500 comprising a bridge 505 and two legs 510 and 511 extending from the first end 501 and the second end 502, respectively. At least the bridge 505 portion of the orthopedic implant 500 is made of a shape memory material such that the bridge is movable between an insertion shape (i.e., deformed shape) and an implanted shape (i.e., the memorized shape). In FIG. 3A, the bridge 505 is in its insertion shape where the bridge 505 is straight. When the shape memory material staple 500 reaches an activation temperature after being implanted into the patient, the bridge 505 returns to its weightbearing implanted shape which makes the bridge 505 to bow upward (in other words, in a direction opposite from the extension direction of the first and second legs 510, 511) as shown in FIG. 3B. This bowing urges the two staple legs 510, 511 to toward each other creating the compression load on the bone fusion site between the two staple legs 510, 511.

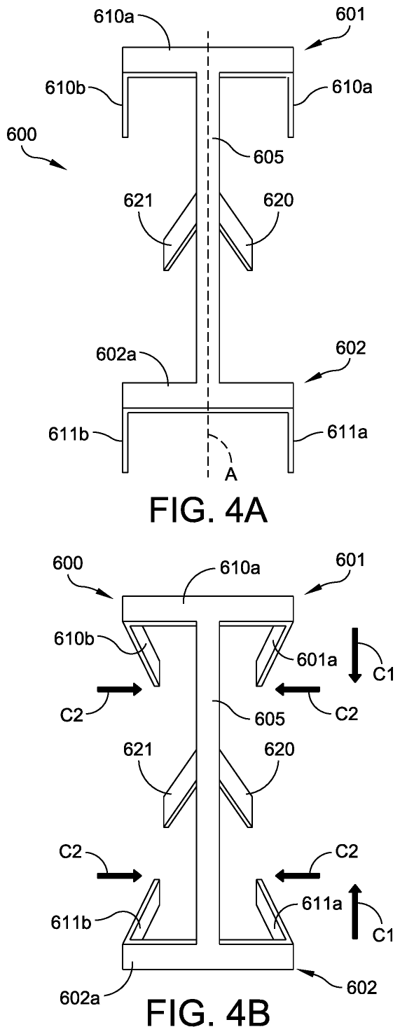

FIGS. 4A-4B show schematic illustrations of a shape memory material orthopedic staple implant that provides multi-planar compression according to some embodiments. The legs of the staples of this embodiment are configured to provide compressive load in multi-direction in addition to the compression in-line/axial with the bridge. Referring to FIG. 4A, such staple 600 comprises a bridge 605 having a first end 601 and a second end 602. Provided at the two ends 601, 602 are bridge extensions 601a and 602a, respectively, that extends the bridge in a transverse direction with respect to the longitudinal axis A of the bridge 605. The transverse direction of the bridge extensions 601a, 602a can be orthogonal to the longitudinal axis A or at some other desired angle depending on the application for the staple 600.

The staple 600 also comprises a plurality of staple legs that extend from the bridge extensions 601a, 602a. In the illustrated example, two staple legs 610a, 610b, and 611a, 611b extend from each of the bridge extensions 601a, 602a. The staple legs 610a and 610b extend from the bridge extension 601a. The staple legs 611a and 611b extend from the bridge extension 602a. The staple 600 can further comprise additional staple legs 620, 621 extending from the bridge 605 between the first and second ends 601, 602. The staple 600 is movable between its insertion shape shown in FIG. 4A and its implanted shape shown in FIG. 4B. As in any shape memory material staple implants, in its insertion shape, all of the legs are oriented to enable insertion of the staple 600 into the prepared holes in a bone fusion site. In its implanted shape shown in FIG. 4B, the legs are being urged in certain desired preset directions to produce compression load in more than one preset directions. In the illustrated example in FIG. 4B, the arrows C1 and C2 show the two different compression directions that are produced by the preconditioning of the staple 600. The legs 610a, 610b, 611a, and 611b in the four corners of the construct of the staple 600 have moved in such a way so that they produce compression in a plane is in-line with the longitudinal axis A of the bridge 605 as represented by the arrows C1. The legs in the four corners of the construct of the staple 600 also produce compression in a plane that is transverse to the longitudinal axis A as represented by the second set of arrows C2. In some embodiments, the transverse direction of the arrows C2 can be orthogonal to the longitudinal axis A. In some embodiments, the transverse direction of the arrows C2 can be at some angle other than orthogonal to the longitudinal axis A.

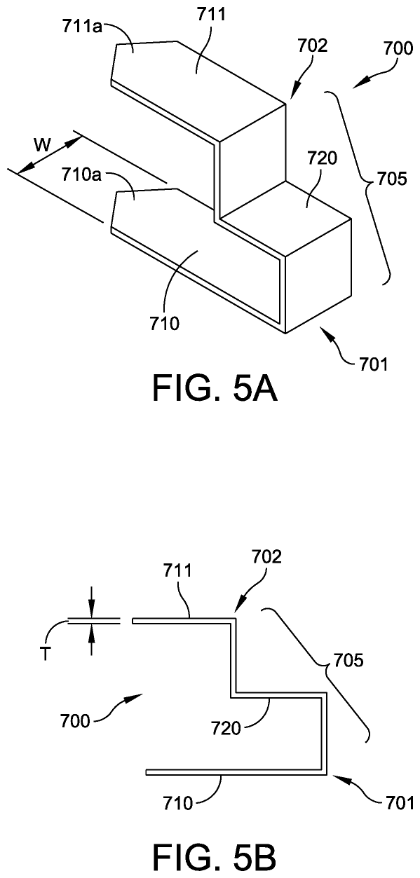

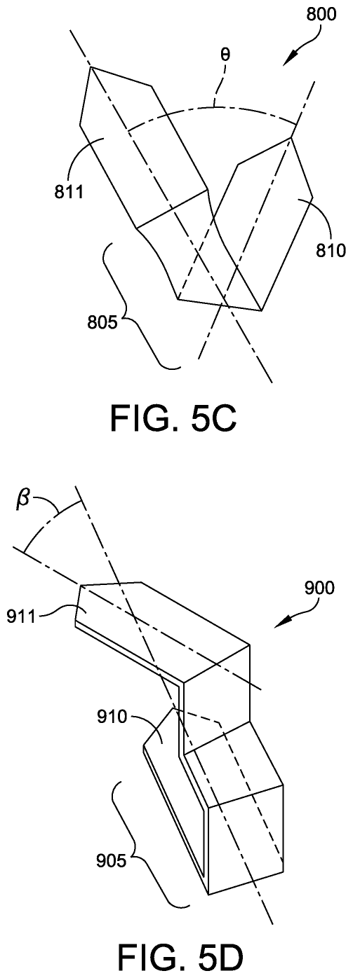

FIGS. 5A-5C show schematic illustrations of a shape memory material orthopedic staple implant that provides improved rotational stability and improved compression in cancellous bone according to some embodiments of the present disclosure. The improved orthopedic staple implant is particularly useful for subtalar fusion. FIG. 5A is an isometric view of an orthopedic staple implant 700 that comprises a bridge 705 having a first end 701 and a second end 702. The staple implant 700 also comprises a first leg 710 extending from the first end 701 and a second leg 711 extending from the second end 702. The legs 710 and 711 extend in an orientation that keeps both legs in-line. The bridge 705 comprises a stepped portion 720. As shown in the side view shown in FIG. 5B, the stepped portion 720 in the bridge 705 allows the first leg 710 and the second leg 711 to be of different length.

Unlike other orthopedic staples, all portions of the orthopedic staple implant 700 comprises a ribbon or blade-like dimension and all portions have a width W that is substantially greater than their thickness T. For purposes of the present disclosure, the width W being “substantially greater” than the thickness T means that the width W is at least twice as wide as the thickness T. Because the width W is substantially greater than the thickness T, that aspect ratio provides the orthopedic staple implant 700 with a greater rotational stability when it is implanted into a bone site. The tips 710a and 711a of the first leg 710 and the second leg 711, respectively can be configured to have a sharp pointed shape to enable easier insertion into the bone material. Because of the greater width W dimension of the orthopedic staple implant 700, multiple holes would be drilled into the receiving bone in-line in order to insert the legs 710, 711 into the bone.

Additionally, compared to the conventional staple implants, the orthopedic staple implant 700 with greater width W would provide a better compression against cancellous bone material because it presents larger load-bearing contact surface area against cancellous bone material.

Referring to FIG. 5C, another embodiment of an orthopedic staple implant 800 whose portions have a width W that is substantially greater than their thickness T is disclosed. The orthopedic staple implant 800 comprises a bridge 805 portion and a first leg 810 and a second leg 811 extending from the two ends of the bridge 805. Unlike the orthopedic staple implant embodiment 700, however, the legs 810 and 811 are not oriented in-line but they extend from the bridge 805 at angles so that their orientation diverge from each other. In the illustrated example of FIG. 5C, the two legs 810 and 811 diverge from each other at an angle θ. This allows the staple 800 to engage two bone pieces on either side of the fusion site whose orientation may not allow staple legs in in-line orientation to engage them. FIG. 5D is a schematic illustration of an orthopedic staple implant 900 according to another embodiment whose portions have a width W that is substantially greater than their thickness T. The staple implant 900 comprises a bridge 905 that has a stepped portion 920 and two legs 910 and 911. The two legs 910 and 911 are oriented to diverge from each other at an angle β that is different from the angle θ. The divergent angles θ and β can be any angle between 0 degree and 180 degrees as needed.

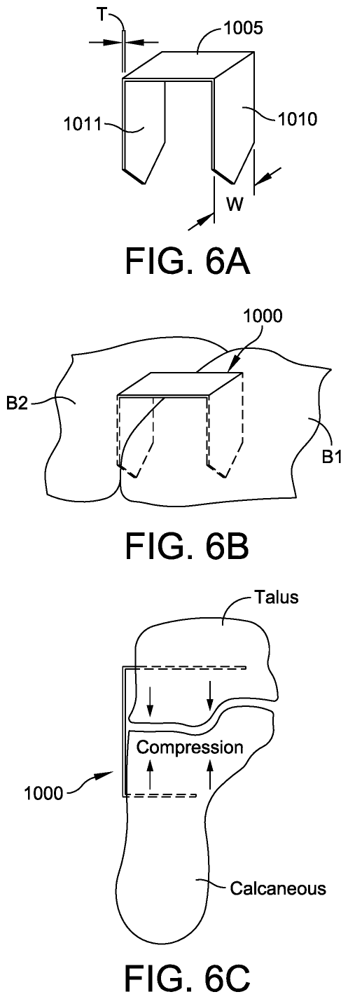

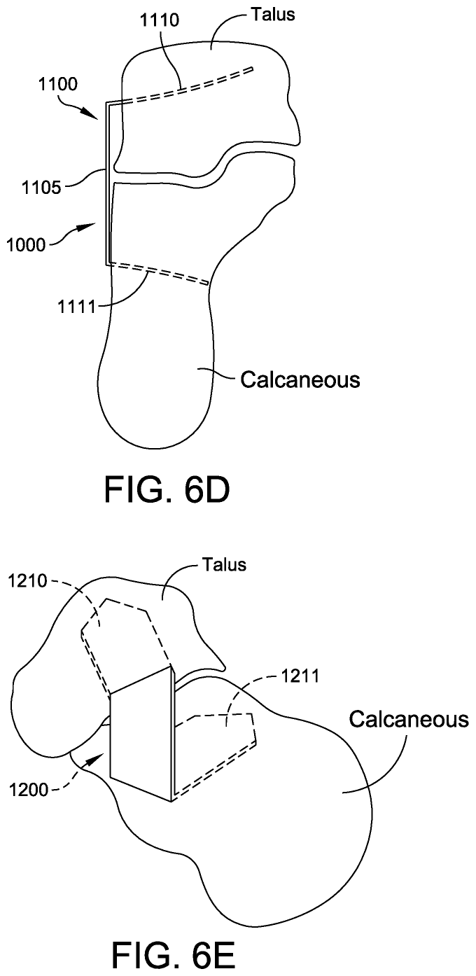

FIGS. 6A-6E are more examples of orthopedic staple implants having ribbon or blade-like dimensions whose width W is substantially greater than its thickness T. FIG. 6A shows an orthopedic staple implant 1000 comprising a bridge 1005 and two legs 1010, 1011 extending from the two ends of the bridge 1005. All portions of the staple implant 1000 has a width W that is substantially greater than their thickness T. FIG. 6B shows the orthopedic staple implant 1000 implanted to compress two bone pieces B1 and B2. Such ribbon or blade-like staple implant 1000 can be useful in a subtalar fusion procedure. FIG. 6C shows the orthopedic staple implant 1000 implanted bridging the talus and calcaneus providing compression load to aid in fusing the talus to the calcaneus. The ribbon or blade-like orthopedic staples 700, 800, 900, 1000 disclosed herein can also be used in many other fusion procedures such as hallux MTP fusion, TN (talus-navicular) fusion, CC (calcaneus-cuboid) fusion, ankle fusion, etc.

FIG. 6D is a side view of an orthopedic staple implant 1100 implanted into a talus and a calcaneus for fusion according to another embodiment. The staple implant 1100 comprises two legs 1110 and 1111 that extend from the bridge 1105. FIG. 6E is a schematic illustration showing a ribbon or blade-like staple implant 1200 implanted in a position engaging the calcaneus and the talar bones. The staple implant 1200 comprises two legs 1210 and 1211 that are not oriented in-line but diverge from each other at an angle that is 1100 is particularly useful for engaging the calcaneus and the talar at locations that cannot be engaged by conventional in-line staple implants.

The instrumentation for implanting the ribbon or blade-like orthopedic staple implants, 700, 800, 900, 1000, 1100, and 1200 can be as follows. Instruments such as a drill peck, a broach, a tap, and an insert device can be useful for preparing the bone compression site and implanting the staple implants.

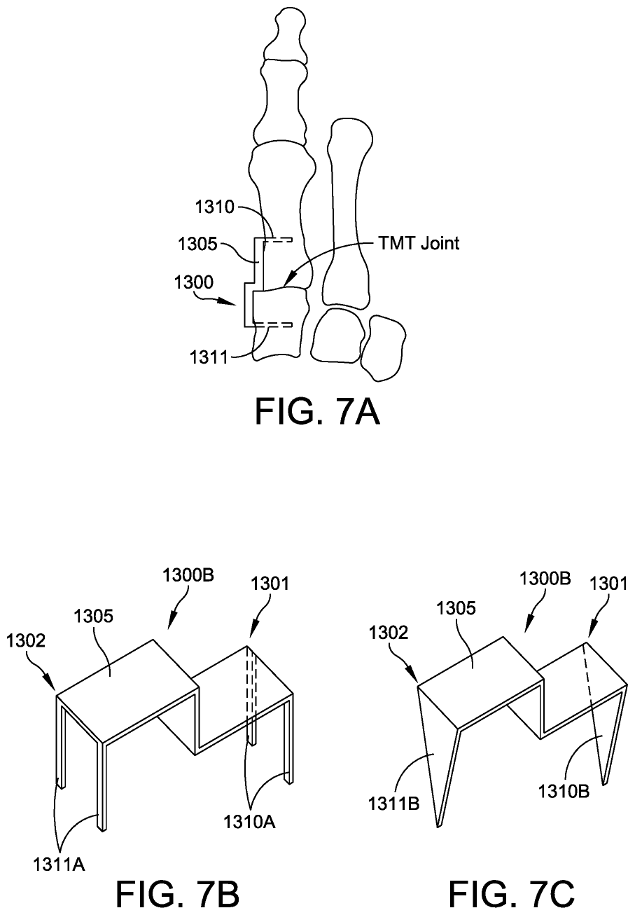

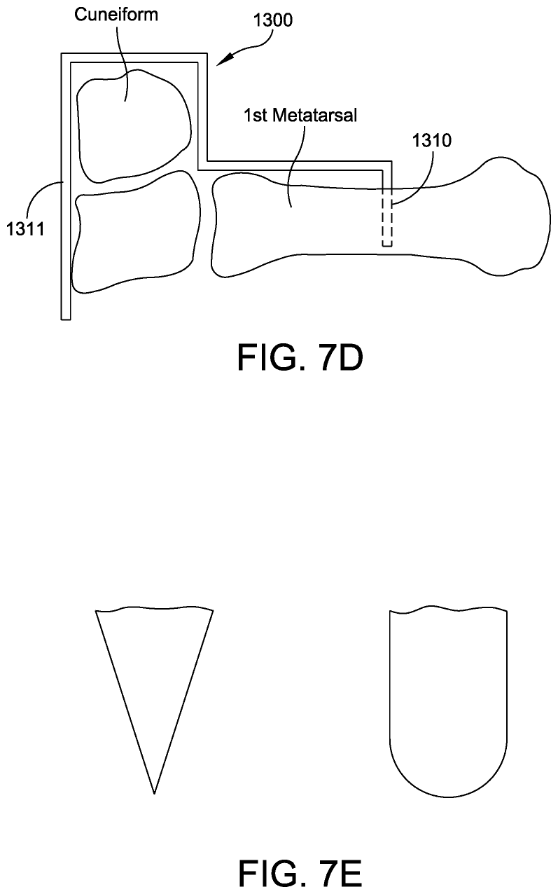

Referring to FIG. 7A, an orthopedic staple implant 1300 whose bridge 1305 portion between the staple legs 1310, 1311 is configured to be more like a bone plate. These staple implants are useful in Lapidus procedures to correct hallux valgus angle and/or intermetatarsal angle to treat bunions. The staple implant has a wide bone plate like bridge 1305 and preferably has two staple legs on each end of the bridge 1305 or has a wide blade-like legs on each end of the bridge. FIGS. 7B and 7C show such variant embodiments. FIG. 7B shows a staple implant 1300A that has a wide plate-like bridge 1305. At the first end 1301 of the bridge are two staple legs 1310A that extend from the bridge 1305. At the second end 1302 of the bridge are two staple legs 1311A that extend from the bridge 1305. FIG. 7C shows a staple implant 1300B that has a wide plate-like bridge 1305. At the first end 1301 of the bridge is a wide blade-like leg 1310B that extend from the bridge. A the second end 1302 of the bridge is a wide blade-like leg 1311B that extend from the bridge. The tips of the blade-like legs 1310B and 1311B can be configured to be blunt or sharp. These two versions are illustrated in FIG. 7E.

The bridge 1305 of the staple implants 1300, 1300A, 1300B is wide like a bone plate and is stepped to accommodate the anatomic step that exists in the TMT joint, for example. FIG. 7D shows a schematic side view of the staple implant 1300 comprising two legs 1310 and 1311 extending from two ends of the bridge. The cuneiform and the first metatarsal bones are shown in dotted lines to illustrate such anatomic step in the TMT joint.

The staple implants 1300, 1300A, 1300B are made of a shape memory material and as such the staple implants are movable between insertion shape and an implanted shape. They are preconditioned to apply compression load to an osteotomy or fracture site when in the implanted shape.

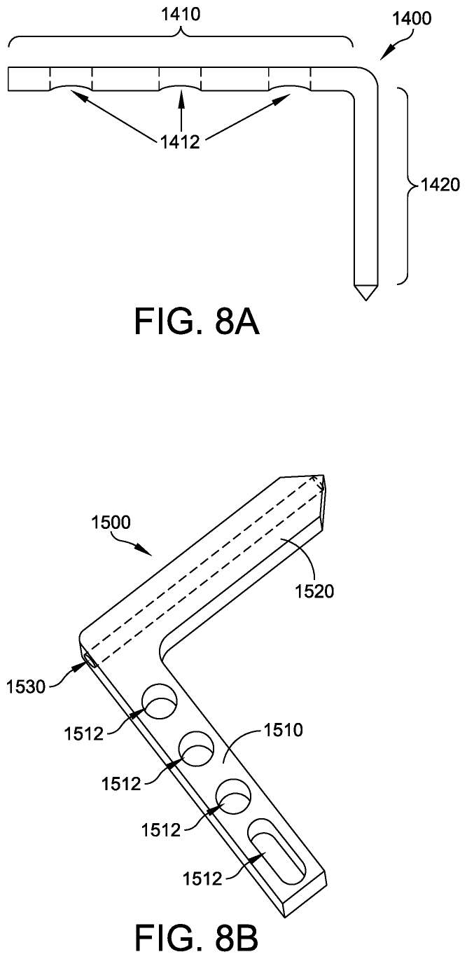

Referring to FIGS. 8A-10I, various examples of blade/plate hybrid orthopedic fixation implant will be disclosed. Such blade/plate hybrid orthopedic fixation implants can be useful in any fusion procedures from hallux MTP, midfoot, hindfoot, to ankle fusions. Referring to FIG. 8A, a blade/plate hybrid orthopedic fixation implant 1400 comprises a plate portion 1410 and a blade portion 1420. The plate portion 1410 comprises one or more screw holes 1412 for receiving bone screws. The screw holes 1412 can be locking, non-locking, or compression type and the plate portion 1410 on one fixation implant 1400 can comprise all, some, or one of the possible types of the screw holes. The blade portion 1420 is similar to the blade-like staple legs 1310B, 1311B on the staple implant embodiment 1300B shown in FIG. 7C.

FIG. 8B shows a blade/plate hybrid orthopedic fixation implant 1500 according to another embodiment. The blade/plate hybrid orthopedic fixation implant 1500 comprises a plate portion 1510 and a blade portion 1520. The plate portion 1510 comprises one or more screw holes 1512 for receiving bone screws. The screw holes 1512 can be locking, non-locking, or compression type and the plate portion 1510 on one fixation implant 1500 can comprise all, some, or one of the possible types of the screw holes. The blade portion 1520 is similar to the blade-like staple legs 1310B, 1311B on the staple implant embodiment 1300B shown in FIG. 7C. However, the blade portion 1520 can be cannulated to receive a guide wire or a fixation pin. As such, the blade portion 1520 comprises a hole 1530 extending through the length of the blade portion 1520.

The blade/plate hybrid orthopedic fixation implant 1500 is made of a shape memory material and as such it is movable between its insertion shape and implanted shape. A possible steps of using the cannulated blade/plate hybrid orthopedic fixation implant 1500 is as follows: (1) place a guide wire into a bone piece for the blade insertion; (2) drill both sides of the guide wire to allow glade insertion; (3) insert the cannulated blade portion 1520 over the guide wire; (4) seat the blade portion 1520 in the bone; (5) ensure that the plate portion 1510 is sitting properly against the bone surface; (6) affix the plate portion 1510 to the bone using bone screws; and (7) allow the blade/plate hybrid orthopedic fixation implant 1500 to move to its implanted shape and apply compression load to the bone fusion site. In some embodiments, the step (3) can be performed using a handle that can control the temperature of the implant to keep the implant maintain its insertion shape. Then, in step (7), the handle is removed to allow the blade/plate hybrid orthopedic fixation implant 1500 to arrive at the patient's body temperature which will enable the implant to move to its implanted shape.

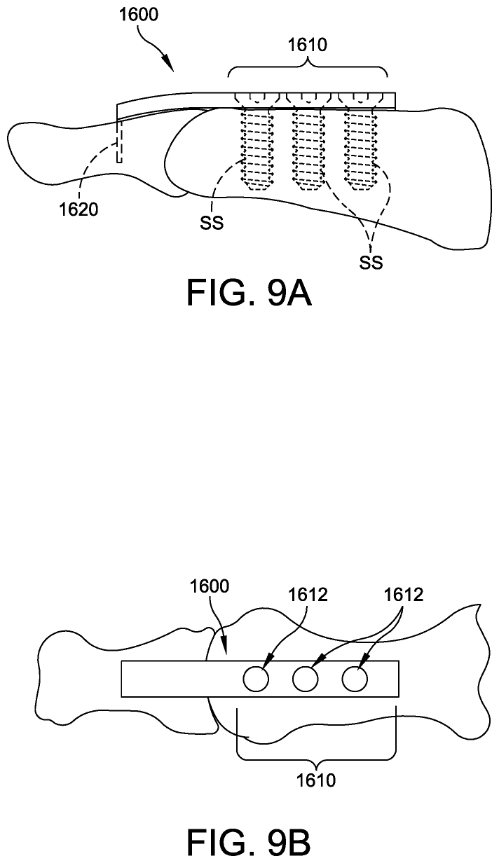

FIGS. 9A-9B show another embodiment of a blade/plate hybrid orthopedic fixation implant 1600. The implant 1600 comprises a plate portion 1610 and a blade portion 1620. The plate portion 1610 comprises one or more screw holes 1612 for receiving bone screws SS. This embodiment is configured to be useful in hallux MP fusion. The blade portion 1620 can be configured to allow some desired degree of dorsiflexion (e.g. 10°) and valgus flexion (e.g. 5°).

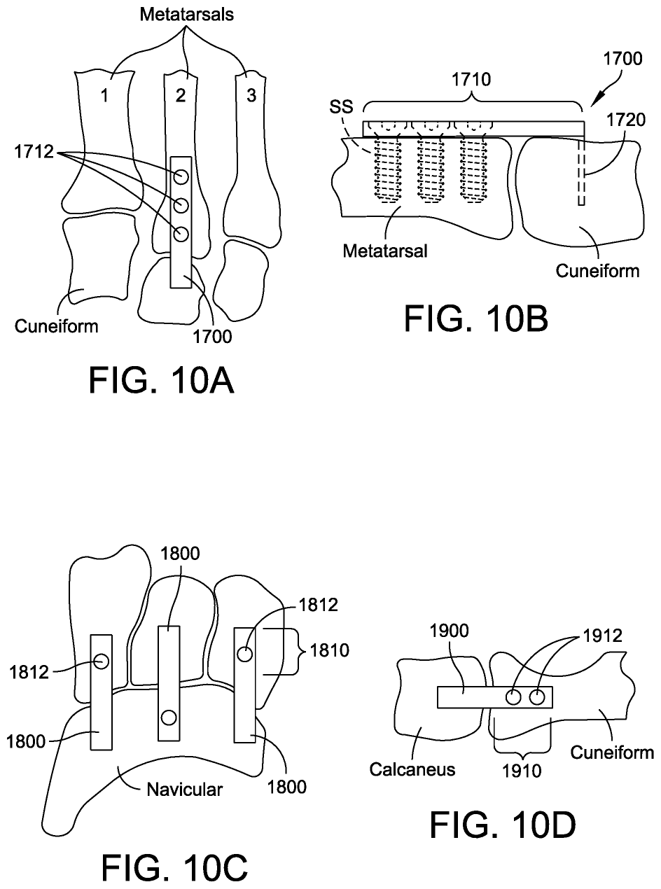

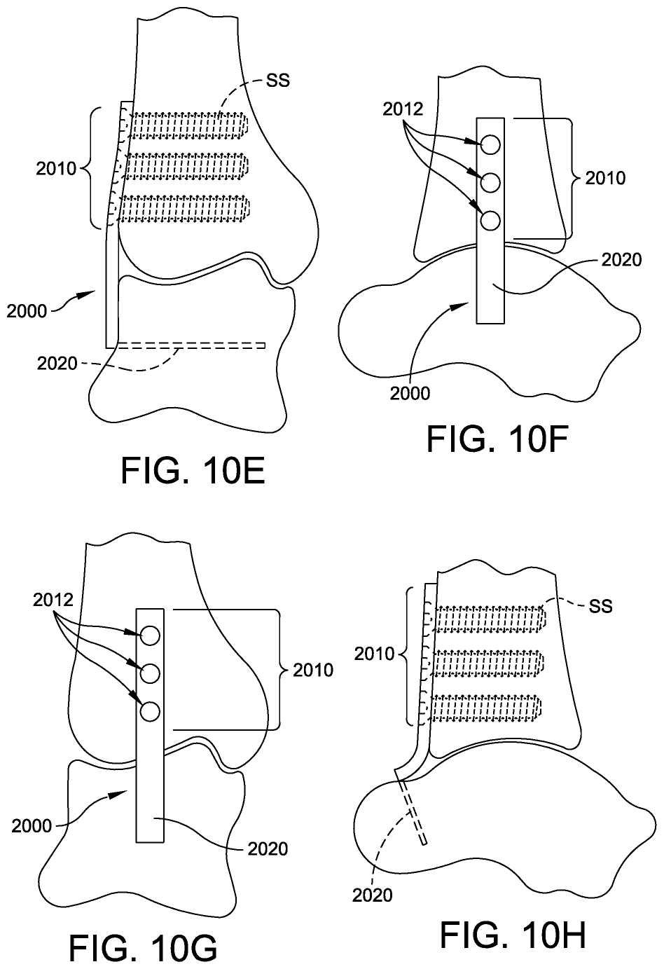

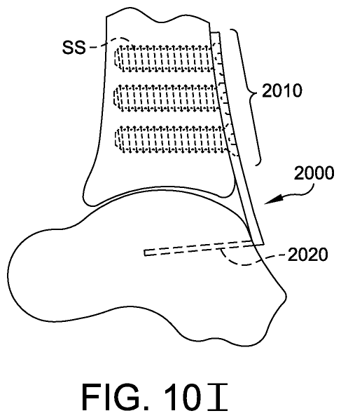

FIGS. 10A-10B show another embodiment of a blade/plate hybrid orthopedic fixation implant 1700. The implant 1700 comprises a plate portion 1710 and a blade portion 1720. The plate portion 1710 comprises one or more screw holes 1712 for receiving bone screws SS. This embodiment is configured to be useful in midfoot, e.g. TMT fusion procedure. Similarly configured implant can also be useful in talonnavicular joint fusion procedure.

FIG. 10C shows another embodiment of a blade/plate hybrid orthopedic fixation implant 1800. The implant 1800 comprises a plate portion 1810 and a blade portion 1820 (not visible in FIG. 10C). The plate portion 1810 comprises one or more screw holes 1812 for receiving bone screws. This embodiment is configured to be useful in navicular-cuneiform fusion procedure.

FIG. 10D shows another embodiment of a blade/plate hybrid orthopedic fixation implant 1900. The implant 1900 comprises a plate portion 1910 and a blade portion 1920 (not visible in FIG. 10D). The plate portion 1910 comprises one or more screw holes 1912 for receiving bone screws. This embodiment is configured to be useful in calcaneous-cuneiform fusion procedure.

FIGS. 10E-10I show another embodiment of a blade/plate hybrid orthopedic fixation implant 2000. The implant 2000 comprises a plate portion 2010 and a blade portion 2020. The plate portion 2010 comprises one or more screw holes 2012 for receiving bone screws. This embodiment is configured to be useful in an ankle fusion procedure. FIGS. 10E-10F show an example of the implant 2000 implantation from the lateral side. FIGS. 10G-10H show an example of the implant 2000 implantation from the anterior side. FIG. 10I shows an example of the implant 2000 implantation from the posterior side. The advantages of the blade/plate hybrid orthopedic fixation implants is that they provide constant compression load similar to staples and also provide the fixation stability of bone plates. Therefore, the blade/plate hybrid fixation implants can be more attractive to those orthopedic surgeons who prefer plates and screws over staples.

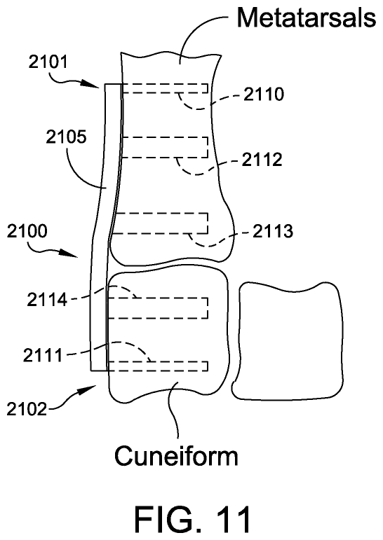

FIG. 11 shows an embodiment of a shape memory material orthopedic fixation implant 2100 that is configured for use in TMT joint fusion procedure. The implant 2100 comprises a bridge 2105 having a first end 2101 and a second end 2102. A first leg 2110 extends from the first end 2101 and a second leg 2111 extends from the second end 2102. The implant 2100 can further comprise additional legs 2112, 2113, and 2114 extending from the bridge 2105 between the first and second legs 2110, 2111. The legs 2111 and 2114 are configured with lengths that are appropriate for the first cuneiform. The legs 2110, 2112, and 2113 are configured with lengths that are appropriate for the first metatasal. The shape of the bridge 2105 is configured to match the slope of the first cunieform. Because the implant 2100 is made of shape memory material, it is movable between an insertion shape and an implanted shape.

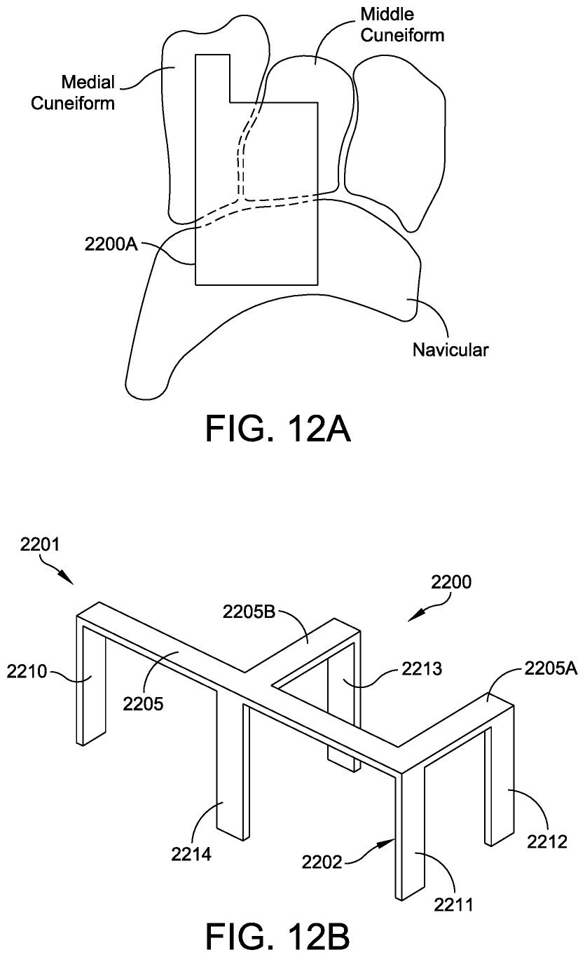

FIGS. 12A-12B show an embodiment of a shape memory material orthopedic fixation implant 2200 that is particularly configured for use in navicular-cuneiform fusion procedure and particularly fusing the medial and middle cuneiforms to the navicular bone. FIG. 12A shows the positions of the navicular, the medial cuneiform, and the middle cuneiform. The outline of the implant 2200 is shown as 2200A illustrating how the implant 2200 is intended to be positioned over the three bones for fusion.

The implant 2200 comprises a primary bridge 2205 that extends from a first end 2201 to a second end 2202 and is sized to extend from the navicular to the medial cuneiform. The first end 2201 comprises two staple legs 2210 and 2214 extending from the primary bridge for inserting into the medial cuneiform. The second end 2202 comprises a staple leg 2211 extending from the primary bridge 2205 for inserting into the navicular. The primary bridge 2205 comprises two secondary bridges 2205A and 2205B that laterally extends from the primary bridge 2205. The secondary bridge 2205A extends laterally and further comprises a staple leg 2212 extending from the secondary bridge 2205A and positioned for inserting into the navicular near the middle cuneiform. The secondary bridge 2205B extends laterally from the primary bridge 2205 toward the middle cuneiform and further comprises a staple leg 2213 extending from the secondary bridge 2205B and positioned for inserting into the middle cuneiform.

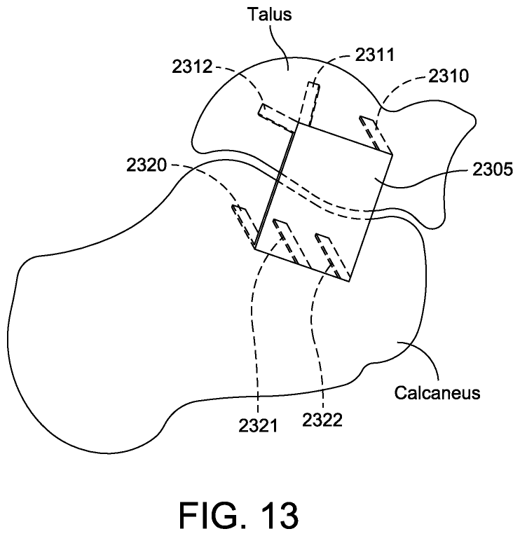

FIG. 13 shows an embodiment of a shape memory material orthopedic fixation implant 2300 that is particularly configured for use in subtalar fusion procedure. The implant 2300 comprises a bridge 2305 that is configured in a pictureframe like shape as shown so that it can cover substantial area between the talus and the calcaneous bones. Extending from the bridge 2305 on one side of the implant 2300 are a plurality of staple legs 2310, 2311, and 2312 for inserting into the talus. In the example shown, three staple legs are provided but the implant 2300 can comprise different number of staple legs for inserting into the talus as necessary. Extending from the bridge 2305 on the opposite side of the implant 2300 are a second set of plurality of staple legs 2320, 2321, and 2322 for inserting into the calcaneus. Again, although only three staple legs are illustrated for inserting into the calcaneus but the implant 2300 can comprise different number of staple legs for inserting into the calcaneus as necessary.

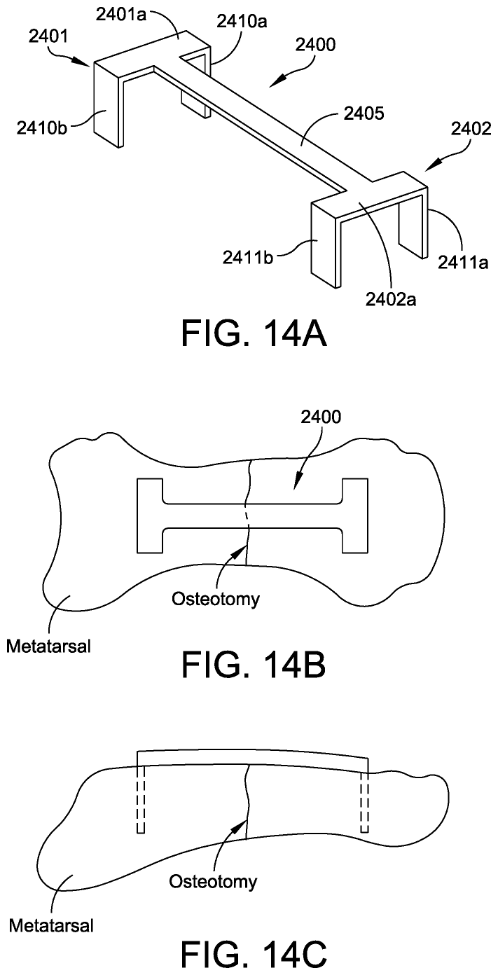

FIGS. 14A-14C show an embodiment of a shape memory material orthopedic fixation implant 2400 that is particularly configured for use in lesser metatarsal osteotomy fusion. FIG. 14A shows a perspective view of the implant 2400 which comprises a bridge 2405 having a first end 2401 and a second end 2402. Provided at the two ends 2401, 2402 are bridge extensions 2401a and 2402a, respectively, that extends the bridge 2405 in a transverse direction with respect to the longitudinal axis of the bridge 2405. The transverse direction of the bridge extensions 2401a, 2402a can be orthogonal to the longitudinal axis or at some other desired angle.

The implant 2400 also comprises a plurality of staple legs that extend from the bridge extensions 2401a, 2402a. In the illustrated example, two staple legs 2410a, 2410b, and 2411a, 2411b extend from each of the bridge extensions 2401a, 2402a. The staple legs 2410a and 2410b extend from the bridge extension 2401a. The staple legs 2411a and 2411b extend from the bridge extension 2402a. Being made of a shape memory material, the implant 2400 is movable between its insertion shape and its implanted shape, the implanted shape being the one that provides the compression load on the osteotomy in the metatarsal shown in FIGS. 14B and 14C.

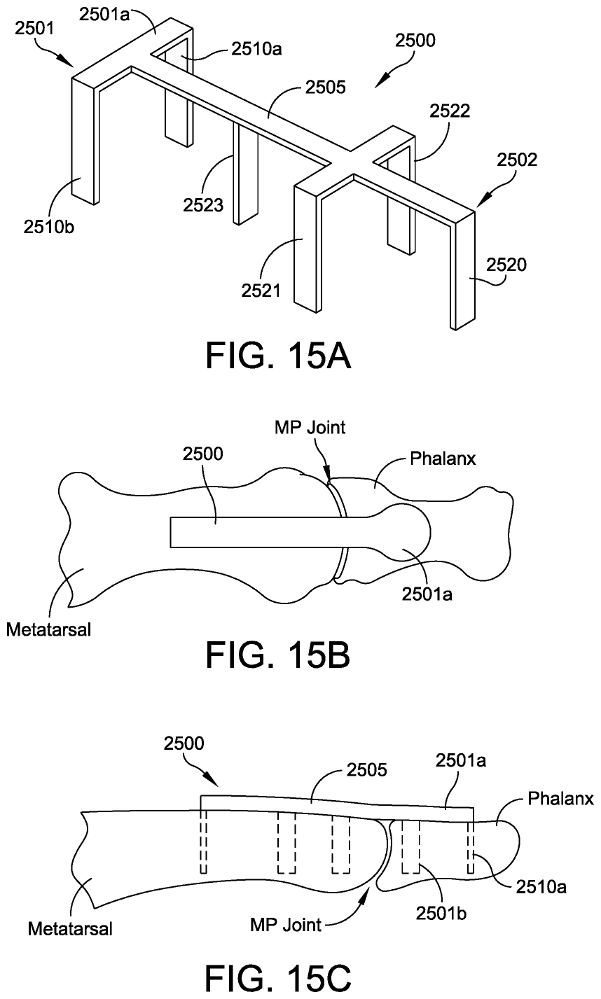

FIGS. 15A-15C show an embodiment of a shape memory material orthopedic fixation implant 2500 that is particularly configured for use in hallux metatarsophalangeal joint fusion procedure. FIG. 15A shows a perspective view of the implant 2500 which comprises a bridge 2505 having a first end 2501 and a second end 2502. Provided at the first end 2501 is a bridge extension 2501a, that extends the bridge 2505 in a transverse direction with respect to the longitudinal axis of the bridge 2505. The transverse direction of the bridge extension 2501a can be orthogonal to the longitudinal axis or at some other desired angle.

The implant 2500 comprises a staple leg 2520 that extend from the second end 2502 and also comprises a plurality of staple legs that extend from the bridge extension 2501a. In the illustrated example, two staple legs 2510a, 2510b extend from the bridge extension 2501a. The implant 2500 also comprises a plurality of additional staple legs 2521, 2522, 2523 that extend from the bridge 2505 between the bridge extension 2501a and the second end 2502. The two staple legs 2510a, 2510b extending from the bridge extension 2501a are configured for inserting into the phalanx whereas the staple legs 2520, 2521, 2522, and 2523 are configured for inserting into the metatarsal.

Being made of a shape memory material, the implant 2500 is movable between its insertion shape and its implanted shape, the implanted shape being the one that provides the compression load to the metatarsophalangeal joint as shown in the anterior-posterior FIGS. 15B and 15C.

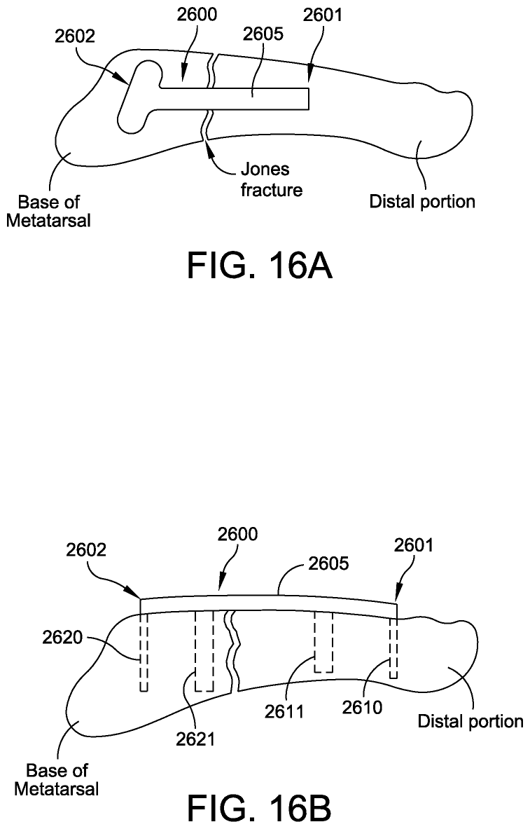

FIGS. 16A and 16B illustrate a shape memory material staple implant 2600 that is particularly configured for fixating a Jones fracture. The implant 2600 comprises a first end 2601 and a second end 2602. The first end 2601 is configured to engage the distal side of the fifth metatarsal around the Jones fracture. The implant 2600 comprises a first staple leg 2610 extending from the first end 2601. The second end 2602 is configured to engage the base portion of the fifth metatarsal around the Jones fracture. The implant 2600 comprises a second staple leg 2620 extending from the second end 2602. The implant 2600 can further comprise additional plurality of staple legs extending from the bridge 2605 between the first leg 2610 and the second leg 2620. In the illustrated exaple, two additional staple legs 2611 and 2621 are provided. The staple leg 2611 closer to the first leg 2610 is configured to be inserted into the distal portion of the fifth metatarsal around the Jones fracture. The staple leg 2621 closer to the second leg 2620 is configured to be inserted into the base portion of the fifth metatarsal.

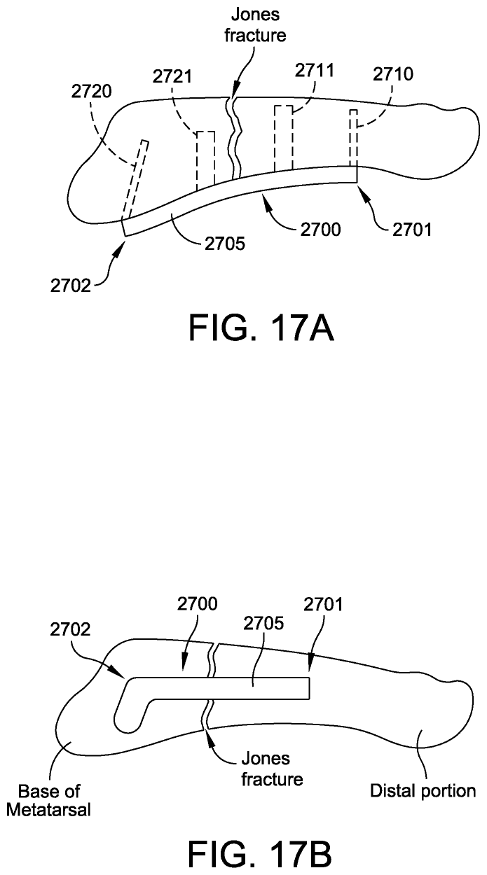

FIGS. 17A and 17B are illustrations of another embodiment of a shape memory material staple implant 2700 that is particularly configured for fixating a Jones fracture. The implant 2700 is similar to the implant 2600 and has a first end 2701 and a second end 2702. The first end 2701 is configured to engage the distal side of the fifth metatarsal around the Jones fracture. The second end 2702 is configured to engage the base portion of the fifth metatarsi around the Jones fracture. The implant 2700 comprises a first staple leg 2710 extending from the first end 2701. The implant 2700 comprises a second staple leg 2720 extending from the second end 2702. The implant 2700 can further comprise additional plurality of staple legs extending from the bridge 2705 between the first leg 2710 and the second leg 2720. In the illustrated example, two additional staple legs 2711 and 2721 are provided. The staple leg 2711 closer to the first leg 2710 is configured to be inserted into the distal portion of the fifth metatarsal around the Jones fracture. The staple leg 2721 closer to the second leg 2720 is configured to be inserted into the base portion of the fifth metatarsal. As shown in the FIGS. 16A and 17B, the second end 2602 and 2702 of the staple implants 2600, 2700 can be configured to match the contour and shape of the base portion of the metatarsal.

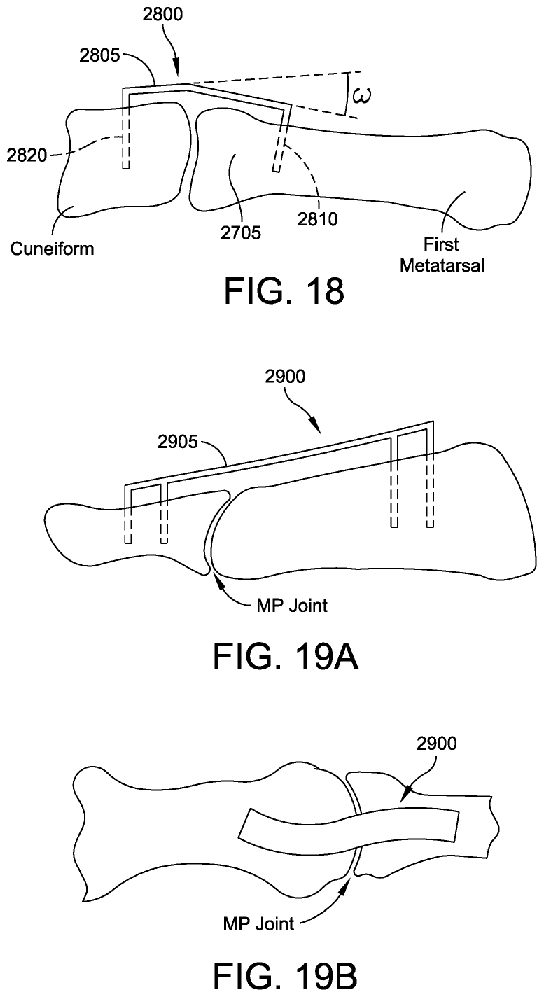

FIG. 18 is an illustration showing another embodiment of a staple implant 2800 that is configured to match the anatomic contour around a TMT joint. The staple implant 2800 comprises a bridge 2805 and a first staple leg 2810 and a second staple leg 2820 extending from the bridge 2805 at its two ends. The bridge 2805 is configured with a 10-20° bend to follow the anatomic contour. The 10-20° bend in the bridge 2805 is identified as the angle co.

FIG. 19A-19B are illustrations of another embodiment of a staple implant 2900 that is configured for hallux metatarsophalangeal fusion. The implant 2900 comprises a bridge 2905 that is curved to match the anatomic contour around hallux metatarsophalangeal (MP) joint. FIG. 19A is a lateral view of the implant 2900 implanted in position over the MP joint. The implant 2900 can be provided with a number of different dorsiflexion angle options for the bridge 2905 to better match the anatomic contour.

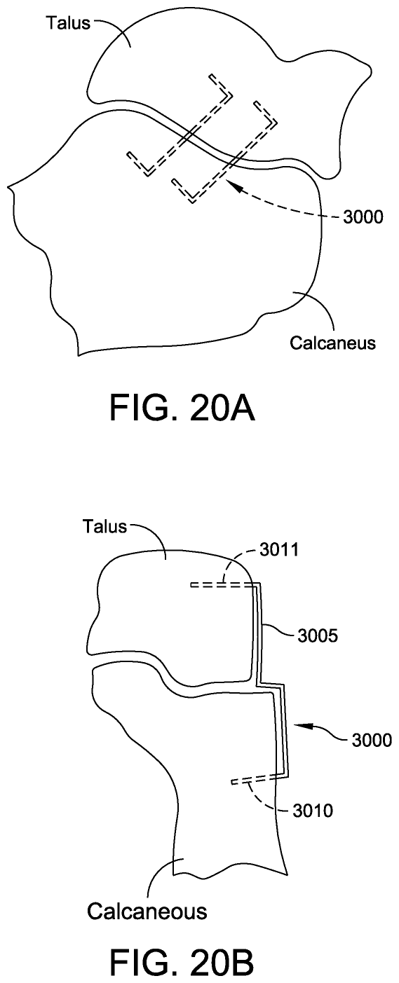

FIGS. 20A-20B are illustrations of a staple implant 3000 according to another embodiment. The implant 3000 is configured to be particularly useful in subtalar fusion procedure. The implant 3000 comprises a bridge 3005 and two staple legs 3010, 3011 extending from the two ends of the bridge 3005. The bridge 3005 is contoured to match the anatomical contour of talus and calcaneus.

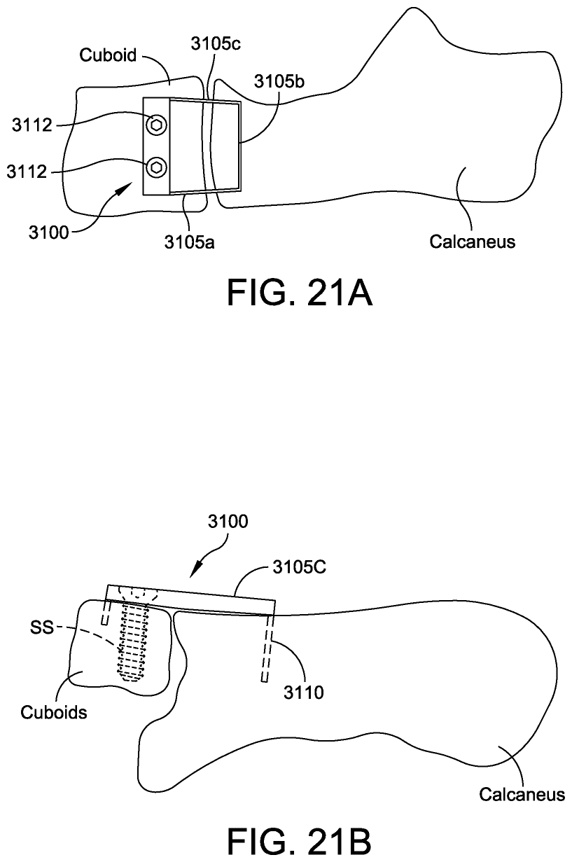

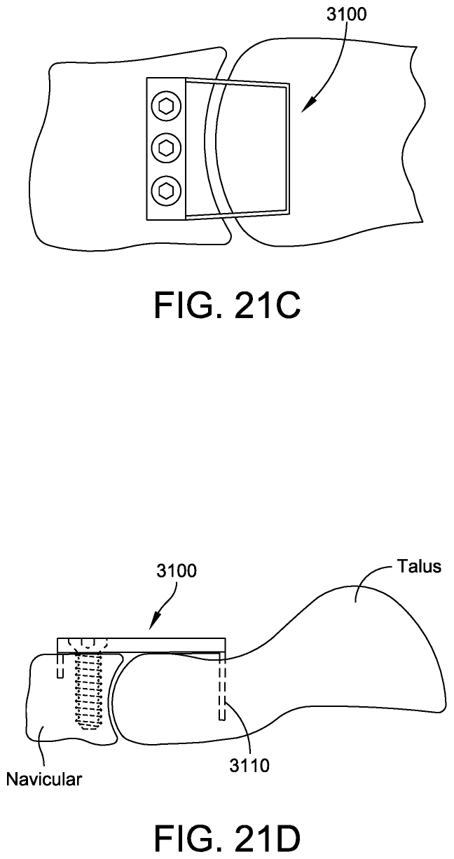

FIGS. 21A-21D are illustrations of bone fixation implant devices that combine a plate structure, suitable for attachment to bone or the like via screws with a Nitinol staple structure that compresses into bone or tissue once released from a holder. The plate structure and the nitinol staple structure are arranged in a coupled side-by-side arrangement. The plate and its screws may be parallel to, perpendicular to, or at another angle to the staple leg(s). The nitinol memory compression may be in the leg(s), the plate, or both. The nitinol leg(s) may be one leg or multiple legs in various alignments and configurations. This surgical device may be used to oppose bone surfaces, whether in fusion or fracture care. The plate/screw interface can be non-locking or locking, or both, and may include compression slots, well known to those of ordinary skill in the art. The plate system includes utility plates (straight, T, L, H), and anatomic plates depending on the area of application.

Referring to FIGS. 21A-21D, additional embodiments of a hybrid implant 3100 comprising a bone plate portion 3115 is disclosed. The plate portion 3115 comprises one or more screw holes 3112 for receiving bone screws SS. The hybrid implant 3100 also comprises a picture frame shaped bridge that comprises at least three segments 3105a, 3105b, and 3105c. FIG. 21A is an illustration showing the hybrid implant 3100 being used to fuse a calcaneus-cuboid joint. The middle segment 3105b of the bridge comprises one or more structures 3110 for inserting into a bone piece. The each of the one or more structures 3110 can be a blade or a staple leg. FIG. 21B shows the side view of the arrangement of FIG. 21A. FIG. 21B shows that the one or more structures 3110 is inserted into the calcaneus. Being made of a shape memory material, the implant 3100 is movable between its insertion shape and its implanted shape, the implanted shape being the one that provides the compression load to the intended fusion site, which in this example is the calcaneus-cuboid joint.

FIG. 21C shows an example of the hybrid implant 3100 being used to fuse the talonavicular joint. FIG. 21D shows a side view of the arrangement of FIG. 21C.

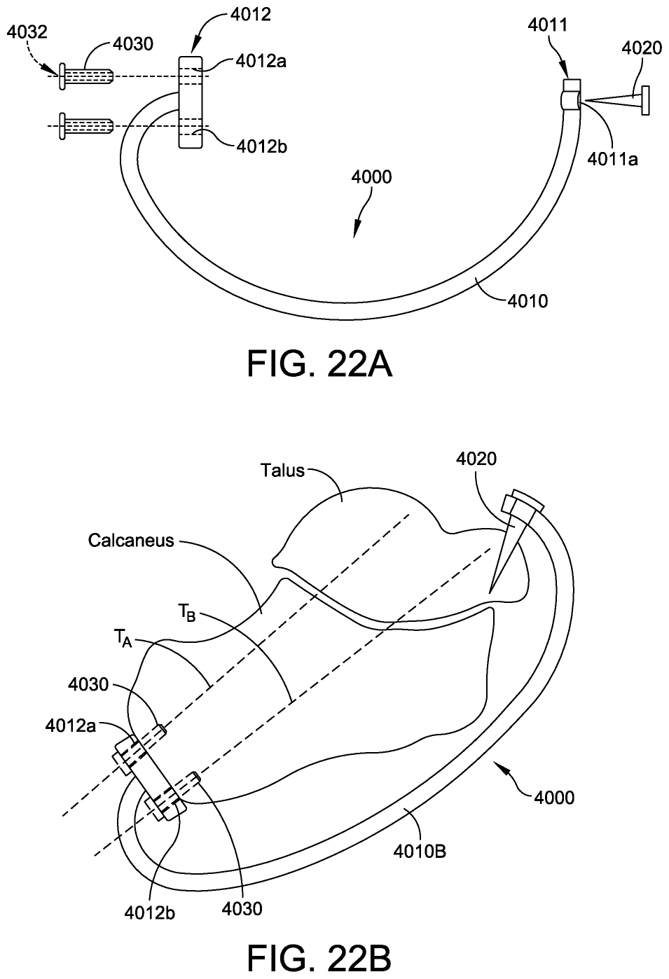

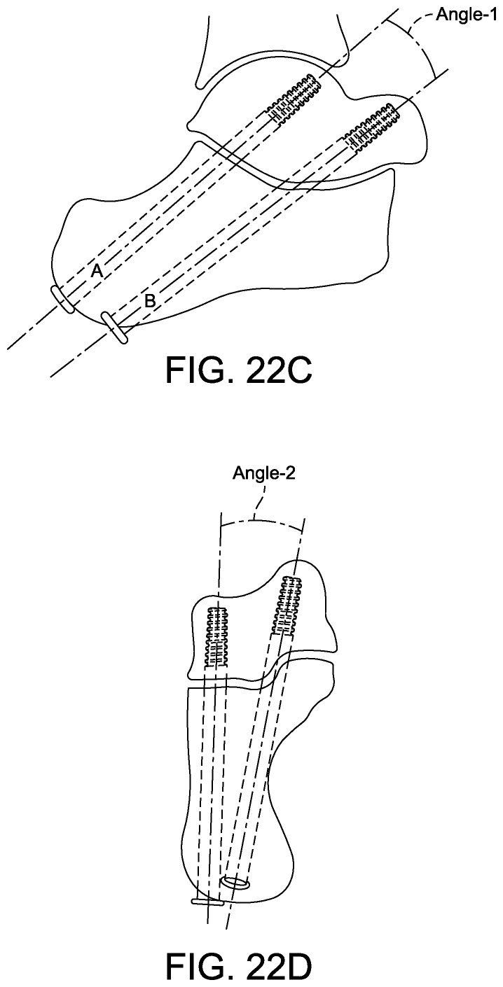

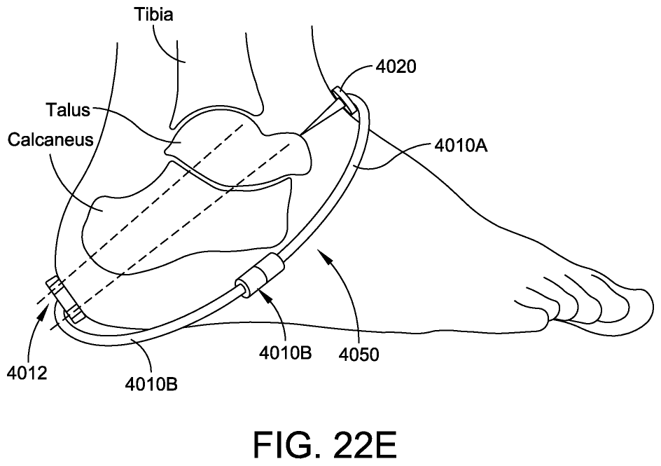

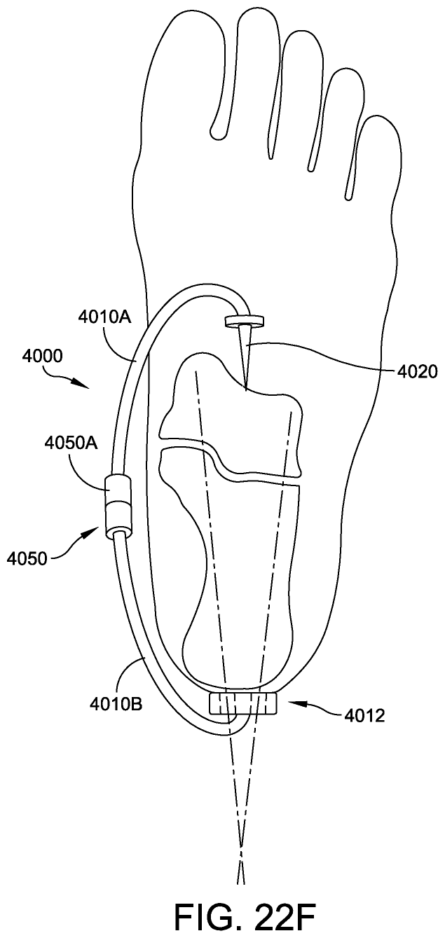

Referring to FIGS. 22A-22F, a subtalar fusion guide 4000 is disclosed. The fusion guide 4000 allows for proper placement of 1 or 2 subtalar fusion screws with better accuracy that allow for the accurate and efficient placement of subtalar fusion screws, eliminating the need for repeated guidewire positioning and repositioning, confirmatory x-rays, etc.

The improved accuracy decreases the length of the surgery and more successful subtalar fusion procedure. One of the desired methods for subtalar fusion is using two bone screws that are screwed into the calcaneus and that talus at an arrangement so that they are double diverging. This means that the two screws A and B shown in FIGS. 22C and 22D diverge from each other in two different directions. In the lateral view of the calcaneus and talus shown in FIG. 22C, the two screws A and B diverge from each other at a first angle Angle-1 in the plantar-dorsal direction. When viewed from the dorsum or plantar side of the foot as shown in FIG. 22D, the two screws A and B diverge from each other at a second angle Angle-2 in the transverse plane. However, properly implanting the two screws A and B in that configuration into the patient's foot can be and is often very consuming and frustrating for the surgeons. The fusion guide 4000 shown in FIGS. 22A-22B, and 22E-22F makes the process of targeting the alignment of the two screws A and B simpler and more accurate.

Referring to FIG. 22A, the fusion guide 4000 comprises a generally C-shaped body 4010 having a first end 4011 and a second end 4012. The body of the fusion guide 4000 is not limited to a C shape as in the example shown. The body of the fusion guide 4000 be in any other shape as desired as long at the two ends 4011 and 4012 are located to be placed around the patient's foot as described. The fusion guide 4000 is configured to be placed around the patient's foot, the first end 4011 being configured to be placed at the talar head and the second end 4012 being configured to be placed at the heel. The first end 4011 comprises a hole 4011a that functions as a sleeve for receiving a drop pin 4020. The second end 4012 comprises two holes 4012a, 4012b that have some depths and their longitudinal axes diverge at the first angle Angle-1 in the plantar-dorsal direction and at the second angle Angle-2 in the transverse plane. The fusion guide 4000 also comprises two cannulated sleeves 4030 that are inserted into the two holes 4012a, 4012b. The cannulated sleeves 4030 each comprises a cannula 4032. The cannula 4032 is sized to receive a guide wire that can be used to guide a drill bit.

When inserted into the holes 4012a, 4012b, the cannulae 4032 of the cannulated sleeves 4030 are oriented such that they define their respective targeting lines TA and TB for the two subtalar fusion screws A and B (shown in FIGS. 22C and 22D). The targeting lines TA and TB diverge from each other in two directions, one at the first angle Angle-1 in the plantar-dorsal direction and a second one at the second angle Angle-2 in the transverse plane. FIG. 22B shows this arrangement. In FIG. 22B, the fusion guide 4000 is placed around a foot with the drop pin 4020 positioned at the talar head and the second end 4012 is positioned at the heel. Two cannulated sleeves 4030 positioned in the holes 4012a, 4012b define the respective targeting lines TA and TB. In a preferred embodiment, the targeting line TB is aimed toward the tip of the drop pin 4020. At this point, the surgeon can check the proper alignment via fluoroscopy, then a guide wire is thrown through the cannulated sleeve 4030 in the second hole 4012b and along the targeting line TB. At this point, the surgeon can check the alignment of the guide wire via fluoroscopy. Next, a second guide wire is thrown through the cannulated sleeve 4030 in the first hole 4012a and along the targeting line TA. Next, holes for the subtalar fusion screws A and B are drilled with a cannulated drill bit using the guide wires as the guide. Next, the fusion guide 4000 assembly is removed and the subtalar fusion screws A and B are screwed into the foot.

In some embodiments of using the fusion guide 4000, aligning of one or both of the screws A and B can be done by freehand if the surgeon determines that is necessary based on the particular patient's anatomy. Rather than using the cannulated sleeve 4030, the surgeon can through the guide wire through the holes 4012a, 4012b in the second end 4012.

Referring to FIGS. 22E and 22F, an embodiment of the fusion guide 4000A comprising a compression feature 4050 is disclosed. In this embodiment, main body of the fusion guide 4000A comprises two portions 4010A and 4010B. The two portions 4010A, 4010B are joined by the compression feature 4050. The two portions 4010A and 4010B are configured so that one of the two portions telescope in and out of the other of the two portions so that the distance between the first end 4011 and the second end 4012 of the fusion guide 4000 can be adjusted. The compression feature 4050 is configured to be able to lock the two portions 4010A, 4010B at a desired position after the desired distance between the two ends of the fusion guide 4000 is achieved.

The locking and unlocking feature of the compression feature 4050 can be enabled by a threaded collet provided on one of the two portions 4010A, 4010B with the other of the two portions 4010A, 4010B received into the threaded collet. The compression feature 4050 can further comprise a ferrule 4050A that slips over the collet and threadedly engage the collet to lock and unlock the telescoping arrangement between the two portions 4010A, 4010B.

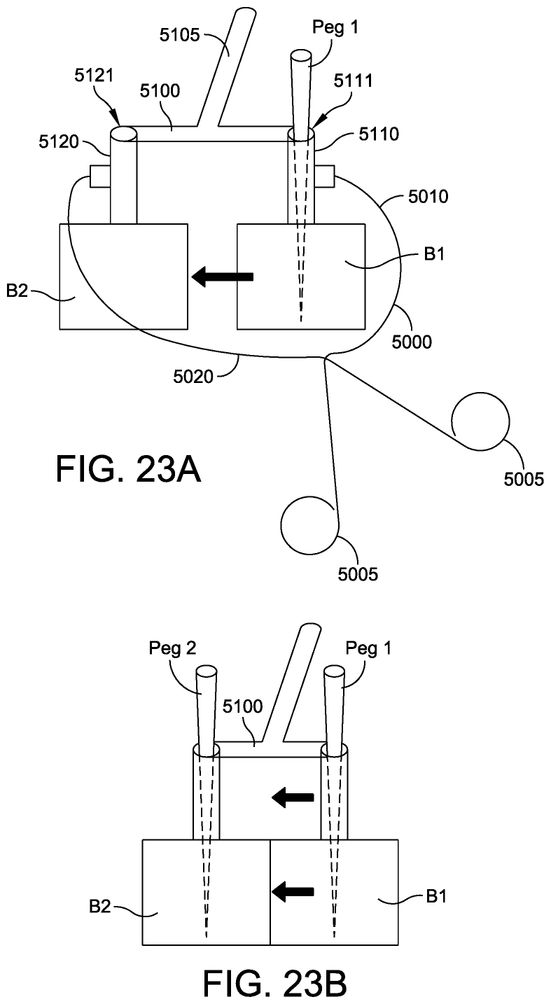

FIGS. 23A-23B are illustrations disclosing a compression device 5000 and a drill guide 5100 for use in implanting orthopedic staples. The drill guide 5100 comprises a handle 5105 and has a bifurcated structure that provides two drill guides 5110 and 5120. The first drill guide 5110 comprises a guide hole 5111. The second drill guide 5120 comprises a guide hole 5121. The drill guide 5100 is configured so that the two drill guides 5110 and 5120 can be urged toward each other to reduce the distance between the two drill guides 5110 and 5120 and the drill guide 5100 will maintain that configuration. The compression device 5000 has a structure like a surgical plier. The compression device 5000 comprises a handle portion 5005 and two compression arms 5010 and 5020. By closing the handle portion 5005 together (as one does with a plier or a pair of scissors), the two compression arms 5010 and 5020 close to apply compression force to the area between the two compression arms 5010 and 5020.

In use, the drill guide 5100 is placed over an osteotomy or a joint between two bone pieces: a distal bone piece B1 and a proximal bone piece B2. The first drill guide 5110 is placed over the distal bone piece B1 and the second drill guide 5120 is placed over the proximal bone piece B2. A hole for receiving a staple leg is first drilled into the distal bone piece B1 through the guide hole 5111 in the first drill guide 5110. A fixation pin or a peg 1 is placed through the guide hole 5111 and into the drilled hole in the distal bone piece B1 as shown in FIG. 23A. Next, the compression device 5000 is used as shown in FIG. 23A to compress the two drill guides 5110 and 5120 together. In this motion, because the first drill guide 5110 is affixed to the distal bone piece B1 by the Peg 1, the distal bone piece B1 will move toward the proximal bone piece B2. The two bone pieces B1, B2 are compressed until they make contact as shown in FIG. 23B. Then, a hole is drilled into the proximal bone piece B2 using the second drill guide 5120 and a second peg 2 is placed into the proximal bone piece B2. Next, the drill guide 5100 and the pegs are removed and an orthopedic staple is placed into the drilled holes, thus holding the two bone pieces B1 and B2 in the compressed state.

Although the devices, kits, systems, and methods have been described in terms of exemplary embodiments, they are not limited thereto. Rather, the appended claims should be construed broadly, to include other variants and embodiments of the devices, kits, systems, and methods, which may be made by those skilled in the art without departing from the scope and range of equivalents of the devices, kits, systems, and methods.

I claim:

1. An orthopedic implant comprising:
a bridge including a first end and a second end;
a first leg extending from the first end of the bridge; and
a second leg extending from the second end of the bridge;
wherein the bridge comprising a metal spacer structure provided in between the first leg and the second leg and extending from the bridge in the same direction as the first and second legs, wherein the metal spacer structure is configured to be inserted between two osteotomy bone surfaces.

2. The orthopedic implant of claim 1, further comprising additional legs provided between the metal spacer structure and the first leg and/or the second leg.

3. The orthopedic implant of claim 1, wherein the metal spacer structure has a porous perforated structure for promoting bone ingrowth into the metal spacer structure after the metal spacer structure is positioned between the two osteotomy bone surfaces.

4. The orthopedic implant of claim 1, wherein the metal spacer structure has a triangular wedge shaped profile and having two planar surfaces facing the first and second legs.

5. The orthopedic implant of claim 1, wherein the metal spacer structure has a trapezoidal shaped profile and having two planar surfaces facing the first and second legs.

6. The orthopedic implant of claim 1, wherein at least the bridge and the legs are made from a shape memory material.

7. An orthopedic implant comprising:
a bridge including a first end and a second end, wherein each of the first and second ends of the bridge is provided with one or more holes for receiving bone screws; and
a first leg and a second leg provided between the first end and the second end and extending from the bridge.

8. The orthopedic implant of claim 7, wherein the holes for the bone screws are threaded to accommodate threaded heads of locking screws.

9. The orthopedic implant of claim 7, wherein at least the bridge and the legs are made from a shape memory material.

10. An orthopedic implant comprising:
a bridge including a first end and a second end;
a first leg extending from the first end of the bridge; and
a second leg extending from the second end of the bridge;
wherein the at least the bridge portion of the orthopedic implant is made of a shape memory material such that the bridge is movable between an insertion shape and an implanted shape, wherein when in the insertion shape the bridge is straight, and when in the implanted shape the bridge is bowed in a direction opposite from the extension direction of the first and second legs and urges the first and second legs toward each other.

11. An orthopedic implant comprising:
a bridge having a longitudinal axis and including a first end and a second end;
a bridge extension provided at each of the first end and the second end, wherein the bridge extensions extend in a transverse direction with respect the longitudinal axis of the bridge; and
a plurality of legs extending from each of the bridge extensions;
wherein the orthopedic implant being made of a shape memory material such that the orthopedic implant is movable between an insertion shape and an implanted shape, wherein in the implanted shape, the plurality of legs are urged in more than one preset directions to produce compression load in the more than one preset directions.

12. The orthopedic implant of claim 11, wherein in the implanted shape, the more than one preset directions comprise at least one direction that produces compression in a plane that is in-line with the longitudinal axis of the bridge.

13. The orthopedic implant of claim 11, wherein in the implanted shape, the more than one preset directions comprise at least one direction that produces compression in a plane that is transverse to the longitudinal axis of the bridge.

14. The orthopedic implant of claim 13, wherein the transverse direction is non orthogonal to the longitudinal axis of the bridge.

15. The orthopedic implant of claim 11, further comprising one or more legs extending from the bridge between the first and second ends.
